(19) United States
(12) Patent Application Publication
(10) Pub. No.: US 2008/0286363 A1
(43) Pub. Date: Nov. 20, 2008

(54) Pharmaceutical Compositions for the Treatment of Inflammatory and Obstructive Airways Diseases

(76) Inventors: Stephen Paul Collingwood, West Sussex (GB); Barbara Haeberlin, Munchenstein (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/094,373

(22) PCT Filed: Nov. 20, 2006
(86) PCT No.: PCT/EP2006/011113

§ 371 Date: May 20, 2008

(30) Foreign Application Priority Data
Nov. 21, 2005 (GB) 0523656.7

(51) Int. Cl. A61K 9/14; A61K 31/58; A61P 29/00; A61P 11/06; A61K 9/12

(52) U.S. Cl. 424/489; 514/171; 424/45; 424/46

(57) ABSTRACT

A medicament comprising, separately or together (A) a compound of formula I

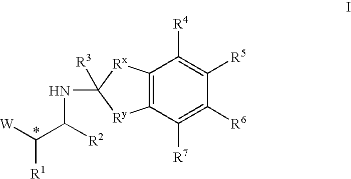

in free or salt or solvate form, (B) a glycopyrronium salt; and (C) a compound of formula II

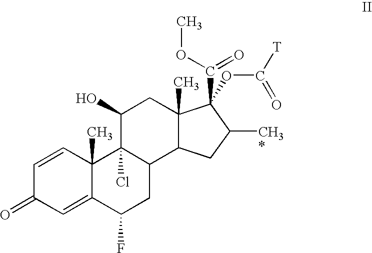

for simultaneous, sequential or separate administration in the treatment of an inflammatory or obstructive airways disease.

This invention relates to organic compounds and their use as pharmaceuticals, in particular for the treatment of inflammatory or obstructive airways diseases.

In a first aspect, the present invention provides a medicament comprising, separately or together
(A) a compound of formula I

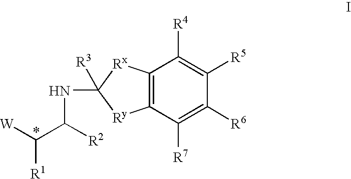

in free or salt or solvate form, wherein
W is a group of formula

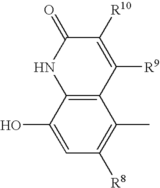

R^{x} and R^{y} are both —CH₂— or —(CH₂)₂—;
R¹ is hydrogen, hydroxy, or C₁-C₁₀-alkoxy;
R² and R³ are each independently hydrogen or C₁-C₁₀-alkyl;
R⁴, R⁵, R⁶ and R⁷ are each independently hydrogen, halogen, cyano, hydroxy, C₁-C₁₀-alkoxy, C₆-C₁₀-aryl, C₁-C₁₀-alkyl, C₁-C₁₀-alkyl substituted by one or more halogen atoms or one or more hydroxy or C₁-C₁₀-alkoxy groups, C₁-C₁₀-alkyl interrupted by one or more hetero atoms, C₂-C₁₀-alkenyl, trialkylsilyl, carboxy, C₁-C₁₀-alkoxycarbonyl, or —CONR¹¹R¹² where R¹¹ and R¹² are each independently hydrogen or C₁-C₁₀-alkyl,
or R⁴ and R⁵, R⁵ and R⁶, or R⁶ and R⁷ together with the carbon atoms to which they are attached denote a 5-, 6- or 7-membered carbocyclic ring or a 4- to 10-membered heterocyclic ring; and
R⁸, R⁹ and R¹⁰ are each independently hydrogen or C₁-C₄-alkyl;
(B) a glycopyrronium salt; and
(C) a compound of formula II

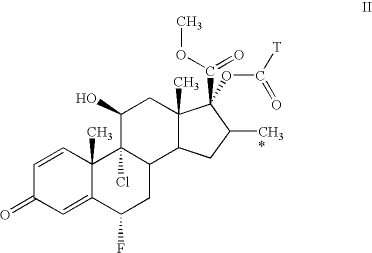

where T is a monovalent cyclic organic group having from 3 to 15 atoms in the ring system;
for simultaneous, sequential or separate administration in the treatment of an inflammatory or obstructive airways disease.

Compounds of formula I in free or salt or solvate form possess beta-2 adrenoceptor agonist activity. They commonly have a rapid onset of action and have a prolonged stimulating action on the β₂-adrenoceptor, for example up 24 hours or longer. They may be prepared by using the procedures described in international patent applications WO 2000/75114, WO 2003/76387, WO 2004/76422 or WO 2004/87668.

Glycopyrronium bromide, or glycopyrrolate, is an antimuscarinic agent that is currently administered by injection to reduce secretions during anaesthesia and or taken orally to treat gastric ulcers. Schroeckenstein et al J. Allergy Clin. Immunol. 1998; 82(1): 115-119 discloses the use of glycopyrrolate in an aerosol formulation for treating asthma where a single administration of a metered dose achieved bronchodilation for up to 12 hours. More recently international patent application WO 2001/76575 discloses glycopyrrolate can be formulated for pulmonary delivery in controlled release formulation that permits the antimuscarinic agent to exert its pharmacological effect over a period greater than 12 hours.

Compounds of formula II are anti-inflammatory corticosteroids that are disclosed in international patent application WO 2002/00679.

It has now surprisingly been found that a significant unexpected therapeutic benefit, particularly a synergistic therapeutic benefit, in the treatment of inflammatory or obstructive airways diseases can be obtained by combination therapy using a compound of formula I in free or salt or solvate form, a glycopyrronium salt and a compound of formula II. For instance, it is possible using this combination therapy to reduce the dosages of one or more of the three active ingredients required for a given therapeutic effect considerably compared with those required using treatment with the active ingredients alone, thereby minimising possibly undesirable side effects. In particular, it has been found that these combinations, particularly compositions containing 5-[2-(5,6-diethyl-indan-2-ylamino)-1-hydroxy-ethyl]-8-hydroxy- 1H-quinolin-2-one maleate, glycopyrronium bromide and a compound of formula II, induce an anti-inflammatory activity which is significantly greater than that induced by 5-[2-(5,6-diethyl-indan-2-ylamino)-1-hydroxy-ethyl]-8-hydroxy-1H-quinolin-2-one maleate, glycopyrronium bromide or a compound of formula II alone. The amount of a compound of formula II in particular needed for a given anti-inflammatory effect may be significantly reduced when used in admixture with 5-[2-(5,6-diethyl-indan-2-ylamino)-1-hydroxy-ethyl]-8-hydroxy-1H-quinolin-2-one maleate and glycopyrronium bromide, thereby reducing the risk of undesirable side effects from the repeated exposure to the steroid involved in the treatment of inflammatory or obstructive airways diseases.

Furthermore, using the combination therapy of the invention, particularly using compositions containing 5-[2-(5,6-diethyl-indan-2-ylamino)-1-hydroxy-ethyl]-8-hydroxy-1H-quinolin-2-one maleate, glycopyrronium bromide and a compound of formula II, medicaments which have a rapid onset of action and a long duration of action may be prepared. Moreover, using such combination therapy, medicaments which result in a significant improvement in lung function may be prepared. Using the combination therapy of the invention, medicaments which provide improved control of obstructive or inflammatory airways diseases, or a reduction in exacerbations of such diseases, may be prepared. Using compositions of the invention, medicaments which can be used on demand in rescue treatment of obstructive or inflammatory airways diseases, or which reduce or eliminate the need for treatment with short-acting rescue medicaments such as salbutamol or terbutaline, may be prepared; thus medicaments based on compositions of the invention facilitate the treatment of an obstructive or inflammatory airways disease with a single medicament.

Accordingly, in a second aspect, the present invention provides a pharmaceutical composition comprising a mixture of effective amounts of (A) as hereinbefore defined, (B) as hereinbefore defined, and (C) as hereinbefore defined, optionally together with at least one pharmaceutically acceptable carrier.

In a third aspect, the present invention provides a method of treating an inflammatory or obstructive airways disease which comprises administering to a subject in need of such treatment effective amounts of (A) as hereinbefore defined, (B) as hereinbefore defined, and (C) as hereinbefore defined.

The invention further provides the use of (A) as hereinbefore defined, (B) as hereinbefore defined, and (C) as hereinbefore defined in the preparation of a medicament for combination therapy by simultaneous, sequential or separate administration of (A), (B) and (C) in the treatment of an inflammatory or obstructive airways disease.

Terms used in the specification have the following meanings:

“C₁-C₁₀-alkoxy” as used herein denotes straight chain or branched alkoxy that contains 1 to 10 carbon atoms.

“C₁-C₁₀-alkyl” as used herein denotes straight chain or branched alkyl that contains one to ten carbon atoms.

“Halogen” or “halo” as used herein denotes an element belonging to group 17 (formerly group VII) of the Periodic Table of Elements, e.g. fluorine, chlorine, bromine or iodine.

“C₆-C₁₀-aryl ” as used herein denotes a monovalent carbocyclic aromatic group that contains 6 to 10 carbon atoms and which may be, for example, a monocyclic group such as phenyl or a bicyclic group such as naphthyl.

“C₂-C₁₀-alkenyl” as used herein denotes straight chain or branched hydrocarbon chains that contain two to ten carbon atoms and one or more carbon-carbon double bonds.

“C₁-C₁₀-alkoxycarbonyl” as used herein denotes C₁-C₁₀-alkoxy as hereinbefore defined linked through an oxygen atom thereof to a carbonyl group.

“5-, 6 or 7-Membered carbocyclic ring” as used herein denotes a carbocyclic group having 5 to 7 ring carbon atoms, either cycloaliphatic, such as a C₅-C₇-cycloalkyl, or aromatic, such as phenyl, which can be substituted by one or more, usually one or two, C₁-C₄-alkyl groups. Where “C₃-C₇-cycloalkyl” denotes cycloalkyl having 3 to 7 ring carbon atoms, for example a monocyclic group such as a cyclopropyl, cyclobutyl, cyclopentyl, cyclohexyl or cycloheptyl, any of which can be substituted by one or more, usually one or two, C₁-C₄-alkyl groups, or a bicyclic group such as bicycloheptyl.

“4- to 10-Membered heterocyclic ring having at least one ring nitrogen, oxygen or sulphur atom” as used herein may be, for example, pyrrole, pyrrolidine, pyrazole, imidazole, triazole, tetrazole, thiadiazole, oxazole, isoxazole, thiophene, thiazole, isothiazole, oxadiazole, pyridine, pyrazine, pyridazine, pyrimidine, piperidine, piperazine, triazine, oxazine, morpholino, quinoline, isoquinoline, naphthyridine, indane or indene.

“C₁-C₄-alkylamino” denotes amino substituted by C₁-C₄-alkyl as hereinbefore defined.

“(Di-C₁-C₄-alkyl)amino” denotes amino disubstituted by C₁-C₄-alkyl as hereinbefore defined.

“Halo-C₁-C₄-alkyl” denotes C₁-C₄-alkyl as hereinbefore defined substituted by one or more, preferably one, two or three halogen atoms, preferably fluorine or chlorine atoms.

“Hydroxy-C₁-C₄-alkyl” denotes C₁-C₄-alkyl as hereinbefore defined substituted by one or more, preferably one, two or three hydroxy groups.

“C₁-C₄-alkylthio” denotes straight chain or branched C₁-C₄-alkylthio and may be methylthio, ethylthio, n-propylthio, isopropylthio, n-butylthio, isobutylthio, sec-butylthio or tert-butylthio.

In one aspect, the present invention provides a medicament comprising, separately or together (A) a compound of formula I as hereinbefore defined in free or salt or solvate form, (B) a glycopyrronium salt, and (C) a compound of formula II as hereinbefore defined for simultaneous, sequential or separate administration in the treatment of an inflammatory or obstructive airways disease.

Compounds of formula I in free or salt or solvate form possess beta-2 adrenoceptor agonist activity. They commonly have a rapid onset of action and have a prolonged stimulating action on the β₂-adrenoceptor, for example up 24 hours or longer.

Preferred compounds of formula I include those wherein R⁸, R⁹ and R¹⁰ are each H, R¹ is OH, R² and R³ are each H and
(i) R^{x} and R^{y} are both —CH₂—, and R⁴ and R⁷ are each CH₃O— and R⁵ and R⁶ are each H;
(ii) R^{x} and R^{y} are both —CH₂—, and R⁴ and R⁷ are each H and R⁵ and R⁶ are each CH₃CH₂—;
(iii) R^{x} and R^{y} are both —CH₂—, and R⁴ and R⁷ are each H and R⁵ and R⁶ are each CH₃—;
(iv) R^{x} and R^{y} are both —CH₂—, and R⁴ and R⁷ are each CH₃CH₂— and R⁵ and R⁶ are each H;
(v) R^{x} and R^{y} are both —CH₂—, and R⁴ and R⁷ are each H and R⁵ and R⁶ together denote —(CH₂)₄—;
(vi) R^{x} and R^{y} are both —CH₂—, and R⁴ and R⁷ are each H and R⁵ and R⁶ together denote —(CH₂)₄—;
(vii) R^{x} and R^{y} are both —CH₂—, and R⁴ and R⁷ are each H and R⁵ and R⁶ are each CH₃(CH₂)₃—;
(viii) R^{x} and R^{y} are both —CH₂—, and R⁴ and R⁷ are each H and R⁵ and R⁶ are each CH₃(CH₂)₂—;
(ix) R^{x} and R^{y} are both —(CH₂)₂—, R⁴, R⁵, R⁶ and R⁷ are each H; or
(x) R^{x} and R^{y} are both —CH₂—, and R⁴ and R⁷ are each H and R⁵ and R⁶ are each CH₃OCH₂—.

Especially preferred compounds of formula I include 8-hydroxy-5-[1-hydroxy-2-(indan-2-yl-amino)-ethyl]-1H-quinolin-2-one, 5-[2-(5,6-dimethoxy-indan-2-ylamino)-1-hydroxy-ethyl]-8-hydroxy-1H-quinolin-2-one, 5-[2-(5,6-diethyl-indan-2-ylamino)-1-hydroxy-ethyl]-8-hydroxy-3-methyl-1H-quinolin-2-one, 5-[2-(5,6-diethyl-indan-2-ylamino)-1-hydroxy-ethyl]-8-methoxy-methoxy-6-methyl-1H-quinolin-2-one, 5-[2-(5,6-diethyl-indan-2-ylamino)-1-hydroxy-ethyl]-8-hydroxy-6-methyl-1H-quinolin-2-one, 8-hydroxy-5-[2-(5,6-diethyl-indan-2-ylamino)-1-hydroxy-ethyl]-3,4-dihydro-1H-quinolin-2-one, 5-[(R)-2-(5,6-diethyl-2-methyl-indan-2-yl-amino)-1-hydroxy-ethyl]-8-hydroxy-1H-quinolin-2-one, (S)-5-[2-(4,7-diethyl-indan-2-ylamino)-1-hydroxy-ethyl]-8-hydroxy-1H-quinolin-2-one hydrochloride, 5-[(R)-1-hydroxy-2-(6,7,8,9-tetrahydro-5H-benzocyclohepten-7-ylamino)-ethyl]-8-hydroxy-1H-quinolin-2-one hydrochloride, (R)-5-[2-(5,6-diethyl-indan-2-ylamino)-1-hydroxy-ethyl]-8-hydroxy-1H-quinolin-2-one maleate, (R)-5-[2-(5,6-diethyl-indan-2-ylamino)-1-hydroxy-ethyl]-8-hydroxy-1H-quinolin-2-one hydrochloride, (R)-8-hydroxy-5-[(S)-1-hydroxy-2-(4,5,6,7-tetramethyl-indan-2-ylamino)-ethyl]-1H-quinolin-2-one, 8-hydroxy-5-[(R)-1-hydroxy-2-(2-methyl-indan-2-ylamino)-ethyl]-1H-quinolin-2-one, 5-[2-(5,6-diethyl-indan-2-ylamino)-ethyl]-8-hydroxy-1H-quinolin-2-one, 8-hydroxy-5-[(R)-1-hydroxy-2-(2-methyl-2,3,5,6,7,8-hexahydro-1H-cyclo-penta[b]naphthalen-2-ylamino)-ethyl]-1H-quinolin-2-one, and 5-[(S)-2-(2,3,5,6,7,8-hexahydro-1H-cyclopenta[b]naph-thalen-2-ylamino)-1-hydroxy-ethyl]-8-hydroxy-1H-quinolin-2-one.

A particularly preferred compound of formula I is a compound of formula III

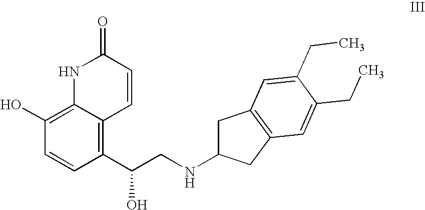

in free or pharmaceutically acceptable salt or solvate form, especially the maleate salt, namely (R)-5-[2-(5,6-diethyl-indan-2-ylamino)-1-hydroxyethyl]-8-hydroxy-1H-quinolin-2-one maleate.

Compounds of formula I in free or salt or solvate form may be prepared by using the procedures described in international patent applications WO 2000/75114, WO 2003/76387, WO 2004/76422 or WO 2004/87668, the contents of which are incorporated herein by reference.

Compounds of formula I or II include all pharmaceutically acceptable isotopically-labelled compounds of formula I or II wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen e.g ²H and ³H, carbon e.g. ¹¹C, ¹³C and ¹⁴C, chlorine e.g. ³⁶Cl, fluorine e.g. ¹⁸F, iodine e.g. ¹²³I and ¹²⁵I, nitrogen e.g. ¹³N and ¹⁵N, oxygen e.g. ¹⁵O, ¹⁷O and ¹⁸O, and sulfur e.g. ³⁵S.

Certain isotopically-labbelled compounds of formula I or II, for example those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with heavier isotopes such as deuterium (²H) may afford certain therapeutic advantages that result from greater metabolic stability, for example increased in vivo half-life or reduced dosage requirements, and hence may be preferred in some circumstances. Substitution with positron emitting isotopes, such as ¹¹C, ¹⁸F, ¹⁵O, and ¹³N can be useful in Positron Emission Topography (PET) studies for examining substrate receptor occupancy.

Isotopically-labelled compounds of formula I or II can generally be prepared by conventional techniques known to those skilled in the art or by processes analogous to those described in the accompanying examples using an appropriate isotopically-labelled reagent in place of the non-labelled reagent previously used.

Compounds of formula I in free form may be converted into salt form, and vice versa, in a conventional manner. The compounds in free or salt form can be obtained in the form of hydrates or solvates containing a solvent used for crystallisation. Compounds of formula I can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Pharmaceutically acceptable salts of the compound of formula I may be acid addition salts, including those of inorganic acids, for example hydrohalic acids such as hydrofluoric acid, hydrochloric acid, hydrobromic acid or hydroiodic acid, nitric acid, sulfuric acid, phosphoric acid; and organic acids such as formic acid, acetic acid, propionic acid, butyric acid, benzoic acid, o-hydroxybenzoic acid, p-hydroxybenzoic acid, p-chlorobenzoic acid, diphenylacetic acid, triphenylacetic acid, 1-hydroxynaphthalene-2-carboxylic acid, 3-hydroxynaphthalene-2-carboxylic acid, aliphatic hydroxy acids such as lactic acid, citric acid, tartaric acid or malic acid, dicarboxylic acids such as fumaric acid, maleic acid or succinic acid, and sulfonic acids such as methanesulfonic acid or benzenesulfonic acid. These salts may be prepared from compounds of formula I by known salt-forming procedures. Pharmaceutically acceptable solvates are generally hydrates.

Pharmaceutically acceptable solvates in accordance with the invention include those wherein the solvent of crystallisation may be isotopically substituted e.g. D₂O, d6-acetone or d₆-DMSO.

Glycopyrronium salts include glycopyrronium bromide, also known as glycopyrrolate, which is known to be an effective antimuscarinic agent. More specifically it inhibits acetyl choline binding to M3 muscarinic receptors thereby inhibiting bronchoconstriction.

Glycopyrrolate is a quaternary ammonium salt. Suitable counter ions are pharmaceutically acceptable counter ions including, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate and benzenesulfonate. Its bromide salt, namely 3-[(cyclopentyl-hydroxyphenylacetyl)oxy]-1,1-dimethylpyrrolidinium bromide, has the following structural formula

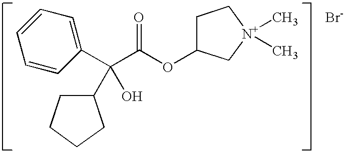

and can be prepared using the procedures described in U.S. Pat. No. 2,956,062.

Glycopyrrolate has two stereogenic centres and hence exists in four isomeric forms, namely (3R,2 R)-, (3S,2 R)-, (3R,2 S)- and (3S,2 S)-3-[(cyclopentyl-hydroxyphenylacetyl)oxy]-1,1-dimethylpyrrolidinium bromide, as described in United States patent specifications U.S. Pat. No. 6,307,060 and U.S. Pat. No. 6,613,795. The contents of these patent specifications are incorporated herein by reference. The present invention embraces using one or more of these isomeric forms, especially the 3S,2 R isomer, the 3R,2 R isomer or the 2S,3′R isomer, thus including single enantiomers, mixtures of diastereomers, or racemates, especially (3S,2 R/3R,2 S)-3-[(cyclopentyl-hydroxyphenylacetyl)oxy]-1,1-dimethylpyrrolidinium bromide.

Compounds of formula II are disclosed, together with procedures for their preparation in international patent application WO 02/00679, the contents of which is incorporated herein by reference. These compounds exhibit surprisingly low systemic side effects at therapeutically effective doses and have a long duration of action, with a potential for once-a-day administration.

In one embodiment, T is a heterocyclic aromatic group having a 5-membered heterocyclic ring with one, two or three ring hetero atoms selected from nitrogen, oxygen and sulfur, the heterocyclic ring being unsubstituted or substituted by one or two substituents selected from halogen, C₁-C₄-alkyl, halo-C₁-C₄-alkyl, C₁-C₄-alkoxy, C₁-C₄-alkyl-thio, cyano or hydroxy-C₁-C₄-alkyl and the heterocyclic ring being optionally fused to a benzene ring. Preferred such heterocyclic aromatic groups include those in which the heterocyclic ring has one nitrogen, oxygen or sulfur atom in the ring or one oxygen and one or two nitrogen atoms in the ring, or one sulfur and one or two nitrogen atoms in the ring, especially a pyrrole, furan, thiophene, oxazole, isoxazole, imidazole, pyrazole, furazan, thiazole or thiadiazole ring. Especially preferred heterocyclic aromatic groups are pyrrolyl, furyl and thienyl groups optionally substituted by one or two substituents selected from halogen (particularly chlorine or bromine), C₁-C₄-alkyl (particularly methyl or ethyl), halo-C₁-C₄-alkyl (particularly trifluoro-methyl), C₁-C₄-alkoxy (particularly methoxy), C₁-C₄-alkylthio (particularly methylthio), cyano or hydroxy-C₁-C₄-alkyl (particularly hydroxymethyl); isoxazolyl, imidazolyl, pyrazolyl, thiazolyl or thiadiazolyl groups optionally substituted by one or two C₁-C₄-alkyl groups; and benzofuryl, benzothienyl and benzofurazanyl groups.

In another embodiment, T is a heterocyclic aromatic group having a 6 membered heterocyclic ring with one, two or three ring heteroatoms, preferably nitrogen, the heterocyclic ring being unsubstituted or substituted by one or more, preferably one, two or three, substituents selected from halogen, cyano, hydroxyl, C₁-C₄-acyloxy, amino, C₁-C₄-alkyl-amino, di-(C₁-C₄-alkyl)amino, C₁-C₄-alkyl, hydroxy-C₁-C₄-alkyl, halo-C₁-C₄-alkyl, C₁-C₄-alkoxy, or C₁-C₄-alkylthio, and the heterocyclic ring being optionally fused to a benzene ring. Preferred such heterocyclic aromatic groups include those in which the heterocyclic group has one or two nitrogen atoms in the ring, especially a pyridine, pyrimidine, pyrazine or pyridazine ring. Especially preferred heterocyclic aromatic groups are pyridyl, pyrimidinyl and pyrazinyl groups, optionally substituted by one or two substituents selected from halogen (particularly chlorine) or C₁-C₄-alkyl (especially methyl or n-butyl).

In compounds of formula II, the indicated methyl group in the 16 position of the cortico-steroid ring system may be in the alpha or beta conformation. 16-α-methyl compounds are preferred.

Especially preferred compounds of formula II are those where the indicated 16-methyl group has the alpha conformation and T is 5-methyl-2-thienyl, N-methyl-2-pyrrolyl, cyclopropyl, 2-furyl, 3-methyl-2-furyl, 3-methyl-2-thienyl, 5-methyl-3-isoxazolyl, 3,5-dimethyl-2-thienyl, 2,5-dimethyl-3-furyl, 4-methyl-2-furyl, 4-(dimethylamino)phenyl, 4-methylphenyl, 4-ethylphenyl, 2-pyridyl, 4-pyrimidyl or 5-methyl-2-pyrazinyl or the indicated 16-methyl group has the beta conformation and R is cyclopropyl.

A particularly preferred compound of formula I is 3-methyl-thiophene-2-carboxylic acid (6S,9R,10S,11S,13S,16R,17R)-9-chloro-6-fluoro-11-hydroxy-17-methoxycarbonyl-10,13,16-trimethyl-3-oxo-6,7,8,9,10,11,12,13,14,15,16,1 7-dodecahydro-3H-cyclopenta-[a]phenanthren-17-yl ester, which has the formula

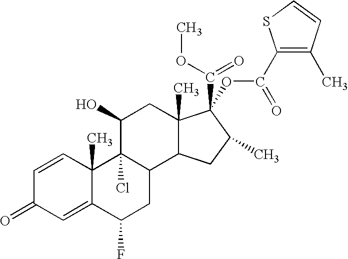

Compounds of formula II in which T contains a basic group are capable of forming acid addition salts, particularly pharmaceutically acceptable acid addition salts. Pharmaceutically acceptable acid addition salts of the compound of formula I include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoroacetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid or triphenylacetic acid, aromatic hydroxy acids such as o-hydroxybenzoic acid, p-hydroxybenzoic acid, 1-hydroxy-naphthalene-2-carboxylic acid or 3-hydroxynaphthalene-2-carboxylic acid, and sulfonic acids such as methanesulfonic acid or benzenesulfonic acid. These salts may be prepared from compounds of formula II by known salt-forming procedures.

Administration of the medicament or pharmaceutical composition as hereinbefore described, i.e. with (A), (B) and (C) in admixture or separate, is preferably by inhalation, i.e. (A), (B) and (C) or the mixture thereof are in inhalable form.

The inhalable form of the medicament may be, for example, an atomizable composition such as an aerosol comprising the active ingredients, i.e. (A), (B) and (C) separately or in admixture, in solution or dispersion in a propellant, or a nebulisable composition comprising a solution or dispersion of the active ingredient in an aqueous, organic or aqueous/organic medium. For example, the inhalable form of the medicament may be an aerosol comprising a mixture of (A), (B) and (C) in solution or dispersion in a propellant, or a combination of an aerosol containing (A) and (B) in solution or dispersion in a propellant with an aerosol containing (C) in solution or dispersion in a propellant. In another example, the inhalable form is a nebulizable composition comprising a dispersion of (A), (B) and (C) in an aqueous, organic or aqueous/organic medium, or a combination of a dispersion of (A) in such a medium with a dispersion of (B) in such a medium and a dispersion of (C) in such a medium.

An aerosol composition suitable for use as the inhalable form of the medicament may comprise the active ingredient in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example chlorine and/or fluorine-substituted methanes, ethanes, propanes, butanes, cyclopropanes or cyclobutanes, such as dichlorodifluoromethane (CFC-12), trichlorofluoromethane (CFC-11), 1,2-dichloro-1,1,2,2-tetrafluoroethane (CFC-114) or, particularly, 1,1,1,2-tetrafluoroethane (HFA-134a), 1,1,1,2,3,3,3-heptafluoropropane (HFA-227), difluorochloromethane (HCFC-22) or mixtures of two or more such halogen-substituted hydrocarbons.

Where the active ingredient is present in suspension in the propellant, i.e. where it is present in particulate form dispersed in the propellant, the aerosol composition may also contain a lubricant and a surfactant, which may be chosen from those lubricants and surfactants known in the art. Other suitable aerosol compositions include surfactant-free or substantially surfactant-free aerosol compositions. The aerosol composition may contain up to about 5% by weight, for example 0.0001 to 5%, 0.001 to 5%, 0.001 to 3%, 0.001 to 2%, 0.001 to 1%, 0.001 to 0.1%, or 0.001 to 0.01%, but preferably 0.01 to 0.5% by weight of the active ingredient, based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurised metered dose inhalation device. The aerosol composition may further contain a bulking agent, for example a sugar such as lactose, sucrose, dextrose, mannitol or sorbitol, in an amount, for example, of up to 20%, usually 0.001 to 1%, by weight of the composition.

In another embodiment of the invention, the inhalable form of the medicament is a dry powder, i.e. (A), (B) and (C) are present in a dry powder comprising finely divided (A), (B) and (C) optionally together with at least one particulate pharmaceutically acceptable carrier, which may be one or more materials known as pharmaceutically acceptable carriers, preferably chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. An especially preferred carrier is lactose, for example lactose monohydrate or anhydrous lactose. The dry powder may be contained as unit doses in capsules of, for example, gelatin or plastic, or in blisters (e.g. of aluminium or plastic), for use in a dry powder inhalation device, which may be a single dose or multiple dose device, preferably in dosage units of (A), (B) and/or (C) together with the carrier in amounts to bring the total weight of powder per capsule to from 5 mg to 50 mg. Alternatively, the dry powder may be contained in a reservoir in a multi-dose dry powder inhalation (MDDPI) device adapted to deliver, for example, 3-25 mg of dry powder per actuation.

In the finely divided particulate form of the medicament, and in the aerosol composition where at least one of the active ingredients are present in particulate form, the active ingredient may have an average particle diameter of up to about 10 μm, for example 0.1 to 5 μm, preferably 1 to 5 μm. The particulate carrier, where present, generally has a maximum particle diameter up to 500 μm, preferably up to 400 μm, and conveniently has a mean particle diameter of 40 to 300 μm, e.g. 50 to 250 μm. The particle size of the active ingredient, and that of a particulate carrier where present in dry powder compositions, can be reduced to the desired level by conventional methods, for example by grinding in an air-jet mill, ball mill or vibrator mill, sieving, microprecipitation, spray-drying, lyophilisation or controlled crystallisation from conventional solvents or from supercritical media.

The inhalable medicament may be administered using an inhalation device suitable for the inhalable form, such devices being well known in the art. Accordingly, the invention also provides a pharmaceutical product comprising a medicament or pharmaceutical composition as hereinbefore described in inhalable form as hereinbefore described in association with one or more inhalation devices. In a further aspect, the invention provides an inhalation device, or a pack of two or more inhalation devices, containing a medicament or pharmaceutical composition as hereinbefore described in inhalable form as hereinbefore described.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992.

Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulised volumes, e.g. 10 to 100 μl, than conventional nebulisers.

Where the inhalable form of the active ingredient is the finely divided particulate form, the inhalation device may be, for example, a dry powder inhalation device adapted to deliver dry powder from a capsule or blister containing a dry powder comprising a dosage unit of (A) and/or (B) or a multi-dose dry powder inhalation (MDDPI) device adapted to deliver, for example, 3-25 mg of dry powder comprising a dosage unit of (A) and/or (B) per actuation. The dry powder composition preferably contains a diluent or carrier, such as lactose, and a compound that helps to protect against product performance deterioration due to moisture e.g. magnesium stearate, typically 0.05-2.0%. Suitable such dry powder inhalation devices are well known. For example, a suitable device for delivery of dry powder in encapsulated form is that described in U.S. Pat. No. 3,991,761, while suitable MDDPI devices include those described in WO 97/20589 and WO 97/30743.

The medicament of the invention is preferably a pharmaceutical composition comprising a mixture of (A) as hereinbefore defined, (B) as hereinbefore defined, and (C) as hereinbefore defined preferably together with at least one pharmaceutically acceptable carrier as hereinbefore described.

A suitable daily dose of the compound (A), the β₂-agonist, for inhalation may be from 20 μg to 2000 μg, for example from 20 to 1500 μg, from 20 to 1000 μg, preferably from 50 to 800 μg, e.g. from 100 to 600 μg or from 100 to 500 μg.

A suitable daily dose of the compound (B), particularly as the bromide salt, for inhalation may be from 10 μg to 2000 μg, preferably from 20 to 1000 μg, and especially from 20 to 800 μg, e.g. from 100 to 500 μg.

A suitable daily dose of the compound (C), a compound of formula II, for inhalation may be from 50 to 2000 μg, for example from 100 to 2000 μg, from 100 to 1600 μg, from 100 to 1000 μg, or from 100 to 800 μg, preferably from 200 to 500 μg, for instance from 200 to 400 μg.

A suitable unit dose of the compound (A), the β₂-agonist, for inhalation may be from 20 μg to 2000 μg, for example from 20 to 1500 μg, from 20 to 1000 μg, preferably from 50 to 800 μg, e.g. from 100 to 600 μg or from 100 to 500 μg.

A suitable unit dose of the compound (B), particularly as the bromide salt, for inhalation may be from 10 μg to 2000 μg, preferably from 20 to 1000 μg, and especially from 20 to 800 μg, e.g. from 100 to 500 μg.

A suitable unit dose of the compound (C), a compound of formula II, for inhalation may be from 50 to 2000 μg, for example from 100 to 2000 μg, from 100 to 1600 μg, from 100 to 1000 μg, or from 100 to 800 μg, preferably from 200 to 500 μg, for instance from 200 to 400 μg.

These unit doses may be administered once or twice daily in accordance with the daily doses mentioned hereinbefore. A single dose is preferred as this is convenient for the patient and encourages compliance. The precise doses of (A), (B) and (C) used will of course depend on the condition to be treated, the patient and the efficiency of the inhalation device.

In one preferred embodiment of the invention, the medicament of the invention is a pharmaceutical composition which is a dry powder in a capsule containing unit doses of (A), (B) and (C), for example for inhalation from a single capsule inhaler, the capsule suitably containing a unit dose of (A) e.g. as hereinbefore described, a unit dose of (B), e.g. as hereinbefore described, and a unit dose of (C), e.g. as hereinbefore described, together with a pharmaceutically acceptable carrier as hereinbefore described in an amount to bring the total weight of dry powder per capsule to between 5 mg and 50 mg, for example 5 mg, 10 mg, 15 mg, 20 mg, 25 mg, 30 mg, 35 mg, 40 mg, 45 mg or 50 mg.

In another preferred embodiment of the invention, the medicament of the invention is a pharmaceutical composition which is a dry powder for administration from a reservoir of a multi-dose dry powder inhaler adapted to deliver, for example, 3 mg to 25 mg of powder containing a unit dose of (A), (B) and (C) per actuation.

In a further preferred embodiment of the invention, the medicament of the invention is a pharmaceutical composition which is an aerosol comprising (A), (B) and (C) as hereinbefore described in a propellant as hereinbefore described, optionally together with a surfactant and/or a bulking agent and/or a co-solvent such as ethanol as hereinbefore described, for administration from a metered dose inhaler adapted to deliver an amount of aerosol containing a unit dose of (A), a unit dose of (B), a unit dose of (C), or a known fraction of a unit dose of (A), a known fraction of a unit dose of (B), and a known fraction of a unit dose of (C) per actuation. Thus if, for example, the inhaler delivers half of the unit doses of (A), (B) and (C) per actuation, the unit doses can be administered by two actuations of the inhaler.

In accordance with the above, the invention also provides a pharmaceutical kit comprising (A), (B) and (C) as hereinbefore defined in separate unit dosage forms, said forms being suitable for administration of (A), (B) and (C) in effective amounts. Such a kit suitably further comprises one or more inhalation devices for administration of (A), (B) and (C). For example, the kit may comprise one or more dry powder inhalation devices adapted to deliver dry powder from a capsule, together with capsules containing a dry powder comprising a dosage unit of (A), capsules containing a dry powder comprising a dosage unit of (B) and capsules containing a dry powder comprising a dosage unit of (C). In another example, the kit may comprise a multi-dose dry powder inhalation device containing in the reservoir thereof a dry powder comprising (A), a multidose dry powder inhalation device containing in the reservoir thereof a dry powder comprising (B) and a multi-dose dry powder inhalation device containing in the reservoir thereof a dry powder comprising (C). In another example, the kit may comprise a multidose dry powder inhalation device containing in the reservoir thereof a dry powder comprising (A) and a multidose dry powder inhalation device containing in the reservoir thereof a dry powder comprising a mixture of (B) and (C). In a yet further example, the kit may comprise a metered dose inhaler containing an aerosol comprising (A) in a propellant, a metered dose inhaler containing an aerosol comprising (B) in a propellant, and a metered dose inhaler containing an aerosol comprising (C) in a propellant.

Medicaments of the invention are advantageous in the treatment of inflammatory or obstructive airways disease, exhibiting highly effective bronchodilatory and anti-inflammatory properties. For instance, it is possible using the combination therapy of the invention to reduce the dosages of corticosteroid required for a given therapeutic effect compared with those required using treatment with a corticosteroid alone, thereby minimising possibly undesirable side effects. In particular, these combinations, particularly where (A), (B) and (C) are in the same composition, facilitate achievement of a high anti-inflammatory effect, such that the amount of corticosteroid needed for a given anti-inflammatory effect may be reduced when used in admixture with (A) and (B), thereby reducing the risk of undesirable side effects from the repeated exposure to the steroid involved in the treatment of inflammatory or obstructive airways diseases. Furthermore, using the combinations of the invention, particularly using compositions containing (A), (B) and (C), medicaments which have a rapid onset of action and a long duration of action may be prepared. Moreover, using such combination therapy, medicaments which result in a significant improvement in lung function may be prepared. In another aspect, using the combination therapy of the invention, medicaments which provide effective control of obstructive or inflammatory airways diseases, or a reduction in exacerbations of such diseases, may be prepared. In a further aspect, using compositions of the invention containing (A), (B) and (C), medicaments which reduce or eliminate the need for treatment with short-acting rescue medicaments such as salbutamol or terbutaline, may be prepared; thus compositions of the invention containing (A), (B) and (C) facilitate the treatment of an obstructive or inflammatory airways disease with a single medicament.

Treatment of inflammatory or obstructive airways diseases in accordance with the invention may be symptomatic or prophylactic treatment. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. cortico-steroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), adult or acute respiratory distress syndrome (ARDS), chronic obstructive pulmonary, airways or lung disease (COPD, COAD or COLD), including chronic bronchitis and emphysema, bronchiectasis and exacerbation of airways hyperreactivity consequent to other drug therapy, in particular other inhaled drug therapy. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tobacosis and byssinosis.

The medicament of the present invention may additionally contain one or more co-therapeutic agents such as anti-inflammatory, bronchodilatory, antihistamine, decongestant or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs.

Co-therapeutic agents include A_{2A} agonists, A_{2B} antagonists, antihistamines, beta-2 adrenoceptor agonists, caspase inhibitors, LTB4 antagonists, LTD4 antagonists, PDE4 inhibitors, mucolytics, matrix metal loproteinase inhibitors (MMPi's), leukotrienes, antibiotics, anti neoplastics, peptides, vaccines, nicotine, elastase inhibitors and sodium cromoglycate.

Suitable A2A agonists include those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083.

Suitable A_{2B} antagonists include those described in WO 02/42298 and WO 03/042214.

Suitable antihistamine drug substances include cetirizine hydrochloride, levocetirizine, acetaminophen, clemastine fumarate, promethazine, loratidine, desloratidine, diphenhydramine and fexofenadine hydrochloride, activastine, astemizole, azelastine, dimetinden, ebastine, epinastine, levocabastine, mizolastine and tefenadine as well as those disclosed in WO 03/099807, WO 04/026841 and JP 2004107299.

Suitable caspase inhibitors, including interleukin-IP converting enzyme (ICE) inhibitors, include those that are disclosed in CA 2109646, GB 2,278,276EP 519748, EP 547 699, EP 590 650, EP 628550, EP 644 197, EP 644198, U.S. Pat. No. 5,411,985, U.S. Pat. No. 5,416,013, U.S. Pat. No. 5,430,128, U.S. Pat. No. 5,434,248, U.S. Pat. No. 5,565,430, U.S. Pat. No. 5,585,357, U.S. Pat. No. 5,656,627, U.S. Pat. No. 5,677,283, U.S. Pat. No. 6,054,487, U.S. Pat. No. 6,531,474, US 20030096737, WO 93/05071, WO 93/14777, WO 93/16710, WO 94/00154, WO 94/03480, WO 94/21673, WO 95/05152, WO 95/35308, WO 97/22618, WO 97/22619, WO 98/10778, WO 98/11109, WO 98/11129, WO 98/41232, WO 99/06367, WO 99/65451, WO 01/119373 and WO 03/32918.

Suitable LTB4 antagonists such as BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700 and WO 04/108720.

Suitable LTD4 antagonists such as montelukast, pranlukast, zafirlukast, accolate, SR2640, Wy-48,252, ICI 198615, MK-571, LY-171883, Ro 24-5913 and L-648051.

Suitable PDE4 inhibitors PDE4 inhibitors such as cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(TM) CC-10004 (Celgene), VM554/UM565 (Vernalis), T-440 (Tanabe), KW-4490 (Kyowa Hakko Kogyo), GRC 3886 (Oglemilast, Glenmark), and those described in WO 92/19594, WO 93/19749, WO 93/19750, WO 93/19751, WO 98/18796, WO 99/16766, WO 01/13953, WO 03/39544, WO 03/104204, WO 03/104205, WO 04/000814, WO 04/000839, WO 04/005258, WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607, WO 04/037805, WO 04/063197, WO 04/103998, WO 04/111044, WO 05/012252, WO 05/012253, WO 05/013995, WO 05/030725, WO 05/030212, WO 05/087744, WO 05/087745, WO 05/087749 and WO 05/090345.

While (A) is a beta-2 adrenoceptor agonist, the medicament of the present invention optionally includes one or more other beta-2 adrenoceptor agonists such as include albuterol (salbutamol), metaproterenol, terbutaline, salmeterol, fenoterol, procaterol, and especially, formoterol, carmoterol, GSK159797 and pharmaceutically acceptable salts thereof, as well as those described in EP 147719, EP 1440966, EP 1460064, EP 1477167, EP 1574501, JP 05025045, JP 2005187357, US 2002/0055651, US 2004/0242622, US 2004/0229904, US 2005/0133417, US 2005/5159448, US 2005/5159448, US 2005/171147, US 2005/182091, US 2005/182092, US 2005/209227, US 2005/256115, US 2005/277632, US 2005/272769, US 2005/239778, US 2005/215542, US 2005/215590, US 2006/19991, US 2006/58530, WO 93/18007, WO 99/64035, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/16601, WO 04/22547, WO 04/32921, WO 04/33412, WO 04/37768, WO 04/37773, WO 04/37807, WO 04/39762, WO 04/39766, WO 04/45618 WO 04/46083, WO 04/80964, WO 04/087142, WO 04/89892, WO 04/108675, WO 04/108676, WO 05/33121, WO 05/40103, WO 05/44787, WO OS/58867, WO OS/65650, WO 05/66140, WO 05/70908, WO 05/74924, WO 05/77361, WO 05/90288, WO 05/92860, WO 05/92887, WO 05/90287, WO OS/95328, WO 05/102350, WO 06/56471, WO 06/74897 or WO 06/8173.

While (B) the glycopyrronium salt is an antimuscarinic agent, the medicament of the present invention optionally includes one or more other antimuscarinic agents such as ipratropium bromide, oxitropium bromide, tiotropium salts, CHF 4226 (Chiesi) and SVT-40776, or those described in EP 424021, U.S. Pat. No. 3,714,357, U.S. Pat. No. 5,171,744, US 2005/171147, US 2005/182091, WO 01/04118, WO 02/00652, WO 02/51841, WO 02/53564, WO 03/00840, WO 03/33495, WO 03/53966, WO 03/87094, WO 04/018422, WO 04/05285, WO 04/96800, WO 05/077361 and WO 06/48225. The medicament of the present invention optionally includes dual beta-2 adrenoceptor agonist / antimuscarinics such as those disclosed in US 2004/0167167, US 2004/0242622, US 2005/182092, US 2005/256114, US 2006/35933, WO 04/74246, WO 04/74812, WO 04/89892 and WO 06/23475.

While (C) a compound of formula II is a steroid, the medicament of the present invention optionally includes one or more other steroids, for example glucocorticosteroids such as budesonide, beclamethasone dipropionate, fluticasone propionate, ciclesonide, mometasone furoate or steroids described in WO 02/88167, WO 02/12266, WO 02/100879, WO 03/35668, WO 03/48181, WO 03/62259, WO 03/64445, WO 03/72592, WO 04/39827 and WO 04/66920, or non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935, WO 04/26248 and WO 05/05452.

The invention is illustrated by the following Examples.

### EXAMPLES

### Compound A1

### (R)-5-[2-(5,6-diethyl-indan-2-ylamino)-1-hydroxy-ethyl]-8-hydroxy-1H-quinolin-2-one maleate

This compound is prepared by reacting (R)-8-benzyloxy-5-oxiranylcarbostyril with 5,6-diethyl-indan-2-ylamine to give 8-benzyloxy-5-[(R)-2-(5,6-diethyl-indan-2-ylamino)-1-hydroxy-ethyl]-IH-quinolin-2-one, subjecting the latter to a deprotecting reaction to replace the benzyl group by hydrogen, and recovering the resultant compound as a maleate salt. Such a process is described in detail in WO 2004/76422, the contents of which is incorporated herein by reference. (R)-8-benzyloxy-5-oxiranylcarbostyril may be prepared as described in WO 1995/25104. 5,6-Diethyl-indan-2-ylamine may be prepared as described in WO 2003/76387.

### Compound B1

### 3-[(Cyclopentyl-hydroxyphenylacetyl)oxy]-1,1-dimethylpyrrolidinium bromide (glycopyrrolate)

This compound is commercially available as a racemate or is prepared using the procedures described in U.S. Pat. No. 2,956,062.

### Compound C

### 3-methyl-thiophene-2-carboxylic acid (6S,9R,10S,11S,13S,16R,17R)-9-chloro-6-fluoro- 11-hydroxy-17-methoxy-carbonyl-10,13,16-trimethyl-3-oxo-6,7,8,9,10,11,12,13,14,15,16,17-dodecahydro-3H-cyclopenta-[a]phenanthren-17-yl ester

This compound is prepared using the procedures described in WO 02/00679.

### Examples 1-60

Gelatin capsules suitable for use in a capsule inhaler such as that described in U.S. Pat. No. 3,991,761 and EP 1270034 are prepared, each capsule containing a dry powder obtained by mixing Compound A1, Compound B1 and Compound C, which have been ground to a mean particle diameter of 1 to 5 μm and lactose monohydrate having a particle diameter below 300 μm, the amounts being as shown in the Table 1 below:

TABLE 1

| | Compound A1 | Compound B1 | Compound C | Lactose |
|---|---|---|---|---|
| Example | (Parts) | (Parts) | (Parts) | (Parts) |
| 1 | 20 | 50 | 50 | 19880 |
| 2 | 40 | 50 | 50 | 19860 |
| 3 | 80 | 50 | 50 | 19820 |
| 4 | 100 | 50 | 50 | 19800 |
| 5 | 120 | 50 | 50 | 19780 |
| 6 | 140 | 50 | 50 | 19760 |
| 7 | 160 | 50 | 50 | 19740 |
| 8 | 180 | 50 | 50 | 19720 |
| 9 | 200 | 50 | 50 | 19700 |
| 10 | 220 | 50 | 50 | 19680 |
| 11 | 240 | 50 | 50 | 19660 |
| 12 | 300 | 50 | 50 | 19600 |
| 13 | 500 | 50 | 50 | 19400 |
| 14 | 1000 | 50 | 50 | 18900 |
| 15 | 2000 | 50 | 50 | 17900 |
| 16 | 20 | 50 | 50 | 24880 |
| 17 | 40 | 50 | 50 | 24860 |
| 18 | 80 | 50 | 50 | 24820 |
| 19 | 100 | 50 | 50 | 24800 |
| 20 | 120 | 50 | 50 | 24780 |
| 21 | 140 | 50 | 50 | 24760 |
| 22 | 160 | 50 | 50 | 24740 |
| 23 | 180 | 50 | 50 | 24720 |
| 24 | 200 | 50 | 50 | 24700 |
| 25 | 220 | 50 | 50 | 24680 |
| 26 | 240 | 50 | 50 | 24660 |
| 27 | 300 | 50 | 50 | 24600 |
| 28 | 500 | 50 | 50 | 24400 |
| 29 | 1000 | 50 | 50 | 23900 |
| 30 | 2000 | 50 | 50 | 22900 |
| 31 | 20 | 100 | 100 | 14780 |
| 32 | 40 | 100 | 100 | 14760 |
| 33 | 80 | 100 | 100 | 14720 |
| 34 | 100 | 100 | 100 | 14700 |
| 35 | 120 | 100 | 100 | 14680 |
| 36 | 140 | 100 | 100 | 14660 |
| 37 | 160 | 100 | 100 | 14640 |
| 38 | 180 | 100 | 100 | 14620 |
| 39 | 200 | 100 | 100 | 14600 |
| 40 | 220 | 100 | 100 | 14580 |
| 41 | 240 | 100 | 100 | 14560 |
| 42 | 300 | 100 | 100 | 14500 |
| 43 | 500 | 100 | 100 | 14300 |
| 44 | 1000 | 100 | 100 | 13800 |
| 45 | 2000 | 100 | 100 | 12800 |
| 46 | 20 | 100 | 100 | 24780 |
| 47 | 40 | 100 | 100 | 24760 |
| 48 | 80 | 100 | 100 | 24720 |
| 49 | 100 | 100 | 100 | 24700 |
| 50 | 120 | 100 | 100 | 24680 |
| 51 | 140 | 100 | 100 | 24660 |
| 52 | 160 | 100 | 100 | 24640 |
| 53 | 180 | 100 | 100 | 24620 |
| 54 | 200 | 100 | 100 | 24600 |
| 55 | 220 | 100 | 100 | 24580 |
| 56 | 240 | 100 | 100 | 24560 |
| 57 | 300 | 100 | 100 | 24500 |
| 58 | 500 | 100 | 100 | 24300 |
| 59 | 1000 | 100 | 100 | 23800 |
| 60 | 2000 | 100 | 100 | 22800 |

### Examples 61-105

A dry powder suitable for delivery from the reservoir of the multi-dose inhaler described in WO 97/20589 is prepared by mixing Compound A1, Compound B1 and Compound C, which have been milled to a mean particle diameter of 1-5 μm and lactose monohydrate having a particle diameter below 300 μm, the amounts being as shown in the Table 2 below:

TABLE 2

| | Compound A1 | Compound B1 | Compound C | Lactose |
|---|---|---|---|---|
| Example | (Parts) | (Parts) | (Parts) | (Parts) |
| 61 | 20 | 50 | 50 | 4880 |
| 62 | 40 | 50 | 50 | 4860 |
| 63 | 80 | 50 | 50 | 4820 |
| 64 | 100 | 50 | 50 | 4800 |
| 65 | 120 | 50 | 50 | 4780 |
| 66 | 140 | 50 | 50 | 4760 |
| 67 | 160 | 50 | 50 | 4740 |
| 68 | 180 | 50 | 50 | 4720 |
| 69 | 200 | 50 | 50 | 4700 |
| 70 | 220 | 50 | 50 | 4680 |
| 71 | 240 | 50 | 50 | 4660 |
| 72 | 300 | 50 | 50 | 4600 |
| 73 | 500 | 50 | 50 | 4400 |
| 74 | 1000 | 50 | 50 | 3900 |
| 75 | 2000 | 50 | 50 | 2900 |
| 76 | 20 | 100 | 100 | 9780 |
| 77 | 40 | 100 | 100 | 9760 |
| 78 | 80 | 100 | 100 | 9720 |
| 79 | 100 | 100 | 100 | 9700 |
| 80 | 120 | 100 | 100 | 9680 |
| 81 | 140 | 100 | 100 | 9660 |
| 82 | 160 | 100 | 100 | 9640 |
| 83 | 180 | 100 | 100 | 9620 |
| 84 | 200 | 100 | 100 | 9600 |
| 85 | 220 | 100 | 100 | 9580 |
| 86 | 240 | 100 | 100 | 9560 |
| 87 | 300 | 100 | 100 | 9500 |
| 88 | 500 | 100 | 100 | 9300 |
| 89 | 1000 | 100 | 100 | 8800 |
| 90 | 2000 | 100 | 100 | 7800 |
| 91 | 20 | 150 | 100 | 14730 |
| 92 | 40 | 150 | 100 | 14710 |
| 93 | 80 | 150 | 100 | 14670 |
| 94 | 100 | 150 | 100 | 14650 |
| 95 | 120 | 150 | 100 | 14630 |
| 96 | 140 | 150 | 100 | 14610 |
| 97 | 160 | 150 | 100 | 14590 |
| 98 | 180 | 150 | 100 | 14570 |
| 99 | 200 | 150 | 100 | 14550 |
| 100 | 220 | 150 | 100 | 14530 |
| 101 | 240 | 150 | 100 | 14510 |
| 102 | 300 | 150 | 100 | 14450 |
| 103 | 500 | 150 | 100 | 14250 |
| 104 | 1000 | 150 | 100 | 13750 |
| 105 | 2000 | 150 | 100 | 12750 |

### Examples 106-135

A dry powder suitable for delivery from the reservoir of the multi-dose inhaler described in WO 97/20589 is prepared by mixing Compound A1, Compound B1 and Compound C, which have been milled to a mean particle diameter of 1-5 μm and lactose monohydrate having a particle diameter below 300 μm, the amounts being as shown in the Table 3 below:

TABLE 3

| | Compound A1 | Compound B1 | Compound C | Lactose |
|---|---|---|---|---|
| Example | (Parts) | (Parts) | (Parts) | (Parts) |
| 106 | 20 | 100 | 200 | 9680 |
| 107 | 40 | 100 | 200 | 9660 |
| 108 | 80 | 100 | 200 | 9620 |
| 109 | 100 | 100 | 200 | 9600 |
| 110 | 120 | 100 | 200 | 9580 |
| 111 | 140 | 100 | 200 | 9560 |
| 112 | 160 | 100 | 200 | 9540 |
| 113 | 180 | 100 | 200 | 9520 |
| 114 | 200 | 100 | 200 | 9500 |
| 115 | 220 | 100 | 200 | 9480 |
| 116 | 240 | 100 | 200 | 9460 |
| 117 | 300 | 100 | 200 | 9400 |
| 118 | 500 | 100 | 200 | 9200 |
| 119 | 1000 | 100 | 200 | 8700 |
| 120 | 2000 | 100 | 200 | 7700 |
| 121 | 20 | 150 | 400 | 14430 |
| 122 | 40 | 150 | 400 | 14410 |
| 123 | 80 | 150 | 400 | 14370 |
| 124 | 100 | 150 | 400 | 14350 |
| 125 | 120 | 150 | 400 | 14330 |
| 126 | 140 | 150 | 400 | 14310 |
| 127 | 160 | 150 | 400 | 14290 |
| 128 | 180 | 150 | 400 | 14270 |
| 129 | 200 | 150 | 400 | 14250 |
| 130 | 220 | 150 | 400 | 14230 |
| 131 | 240 | 150 | 400 | 14210 |
| 132 | 300 | 150 | 400 | 14150 |
| 133 | 500 | 150 | 400 | 13950 |
| 134 | 1000 | 150 | 400 | 13450 |
| 135 | 2000 | 150 | 400 | 12450 |

### Examples 136-180

A dry powder suitable for delivery from the reservoir of the multi-dose inhaler described in WO 97/20589 is prepared by mixing Compound A1, Compound B1 and Compound C, which have been milled to a mean particle diameter of 1-5 μm and lactose monohydrate having a particle diameter below 300 μm, the amounts being as shown in the Table 2 but also containing 0.5% magnesium stearate by weight.

### Examples 181-210

A dry powder suitable for delivery from the reservoir of the multi-dose inhaler described in WO 97/20589 is prepared by mixing Compound A1, Compound B1 and Compound C, which have been milled to a mean particle diameter of 1-5 μm and lactose monohydrate having a particle diameter below 300 μm, the amounts being as shown in the Table 3 but also containing 1% magnesium stearate by weight.

### Examples 211-234

Aerosol formulations are prepared by dispensing micronised active ingredients, Compound A1, Compound B1 and Compound C, and if required, lactose as bulking agent into a vial, sealing the vial with a metering valve, injecting the premixed ethanol/propellant and optional surfactant into the vial through the valve and subjecting the vial to ultrasonic energy to disperse the solid particles. The components and amounts used are shown in Table 4 below, where OA is oleic acid:

TABLE 4

| | Cpd. A1 | Cpd. B1 | Cpd. C | HFA134a | HFA227 | Ethanol | OA | Lactose |
|---|---|---|---|---|---|---|---|---|
| Ex. | (Parts) | (Parts) | (Parts) | (Parts) | (Parts) | (Parts) | (Parts) | (Parts) |
| 211 | 2 | 5 | 5 | 36500 | 60750 | 2500 | — | 70 |
| 212 | 4 | 5 | 5 | 3410 | 6340 | 230 | 0.3 | — |
| 213 | 8 | 5 | 5 | 97000 | — | 2500 | — | 90 |
| 214 | 10 | 5 | 5 | 30500 | 67000 | 2500 | 0.5 | 100 |
| 215 | 12 | 5 | 5 | 3150 | 6550 | 250 | 1 | — |
| 216 | 14 | 5 | 5 | 3700 | 6050 | 250 | 0.8 | — |
| 217 | 16 | 5 | 5 | 3800 | 5900 | 230 | 0.4 | — |
| 218 | 18 | 5 | 5 | 4700 | 5050 | 250 | 1 | — |
| 219 | 20 | 10 | 10 | 3600 | 6150 | 225 | 1 | — |
| 220 | 22 | 10 | 10 | 3500 | 6200 | 230 | 1 | — |
| 221 | 24 | 10 | 10 | 98000 | — | 2500 | 1 | — |
| 222 | 30 | 10 | 10 | 3900 | 5900 | 250 | 1 | — |
| 223 | 2 | 10 | 10 | 30000 | 67000 | 2250 | 0.2 | 90 |
| 224 | 10 | 10 | 10 | 3500 | 6200 | 250 | 0.5 | — |
| 225 | 14 | 10 | 10 | 3200 | 6500 | 230 | 1 | — |
| 226 | 18 | 10 | 10 | 3100 | 6200 | 225 | 0.8 | — |
| 227 | 20 | 10 | 10 | 3150 | 6100 | 225 | 1 | — |
| 228 | 24 | 10 | 10 | 30000 | 60000 | 2000 | 0.8 | — |
| 229 | 2 | 10 | 20 | 30000 | 67000 | 2250 | 0.2 | 90 |
| 230 | 10 | 10 | 20 | 3500 | 6200 | 250 | 0.5 | — |
| 231 | 14 | 10 | 20 | 3200 | 6500 | 230 | 1 | — |
| 232 | 18 | 10 | 20 | 3100 | 6200 | 225 | 0.8 | — |
| 233 | 20 | 10 | 20 | 3150 | 6100 | 225 | 1 | — |
| 234 | 24 | 10 | 20 | 30000 | 60000 | 2000 | 0.8 | — |

1. A medicament comprising, separately or together
(A) a compound of formula I 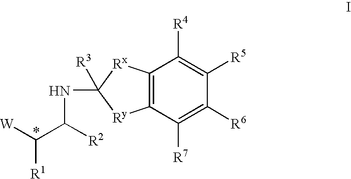
in free or salt or solvate form, wherein
W is a group of formula 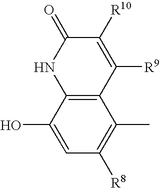
R^{x} and R^{y} are both —CH₂— or —(CH₂)₂—;
R¹ is hydrogen, hydroxy, or C₁-C₁₀-alkoxy;
R² and R³ are each independently hydrogen or C₁-C₁₀-alkyl;
R⁴, R⁵, R⁶ and R⁷ are each independently hydrogen, halogen, cyano, hydroxy, C₁-C₁₀-alkoxy, C₆-C₁₀-aryl, C₁-C₁₀-alkyl, C₁-C₁₀-alkyl substituted by one or more halogen atoms or one or more hydroxy or C₁-C₁₀-alkoxy groups, C₁-C₁₀-alkyl interrupted by one or more hetero atoms, C₂-C₁₀-alkenyl, trialkylsilyl, carboxy, C₁-C₁₀-alkoxycarbonyl, or —CONR¹¹R¹² where R¹¹ and R¹² are each independently hydrogen or C₁-C₁₀-alkyl,
or R⁴ and R⁵, R⁵ and R⁶, or R⁶ and R⁷ together with the carbon atoms to which they are attached denote a 5-, 6- or 7-membered carbocyclic ring or a 4- to 10-membered heterocyclic ring; and
R⁸, R⁹ and R¹⁰ are each independently hydrogen or C₁-C₄-alkyl;
(B) a glycopyrronium salt; and
(C) a compound of formula II 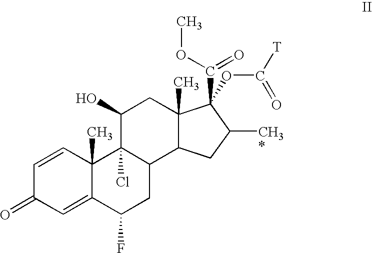
where T is a monovalent cyclic organic group having from 3 to 15 atoms in the ring system; for simultaneous, sequential or separate administration in the treatment of an inflammatory or obstructive airways disease.

2. A medicament according to claim 1 which is a pharmaceutical composition comprising a mixture of effective amounts of (A), (B) and (C) optionally together with at least one pharmaceutically acceptable carrier.

3. A medicament according to claim 1 or 2, in which (A) is a compound of formula I in free or salt or solvate form wherein R⁸, R⁹ and R¹⁰ are each H, R¹ is OH, R² and R³ are each H and
(i) R^{x} and R^{y} are both —CH₂—, and R⁴ and R⁷ are each CH₃O— and R⁵ and R⁶ are each H;
(ii) R^{x} and R^{y} are both —CH₂—, and R⁴ and R⁷ are each H and R⁵ and R⁶ are each CH₃CH₂—;
(iii) R^{x} and R^{y} are both —CH₂—, and R⁴ and R⁷ are each H and R⁵ and R⁶ are each CH₃—;
(iv) R^{x} and R^{y} are both —CH₂—, and R⁴ and R⁷ are each CH₃CH₂— and R⁵ and R⁶ are each H;
(v) R^{x} and R^{y} are both —CH₂—, and R⁴ and R⁷ are each H and R⁵ and R⁶ together denote —(CH₂)₄—;
(vi) R^{x} and R^{y} are both —CH₂—, and R⁴ and R⁷ are each H and R⁵ and R⁶ together denote —O(CH₂)₂O—;
(vii) R^{x} and R^{y} are both —CH₂—, and R⁴ and R⁷ are each H and R⁵ and R⁶ are each CH₃(CH₂)₃—;
(viii) R^{x} and R^{y} are both —CH₂—, and R⁴ and R⁷ are each H and R⁵ and R⁶ are each CH₃(CH₂)₂—;
(ix) R^{x} and R^{y} are both —(CH₂)₂—, R⁴, R⁵, R⁶ and R⁷ are each H; or
(x) R^{x} and R^{y} are both —CH₂—, and R⁴ and R⁷ are each H and R⁵ and R⁶ are each CH₃OCH₂—.

4. A medicament according to claim 1, in which (A) is a compound of formula I selected from the group consisting of 8-hydroxy-5-[1-hydroxy-2-(indan-2-yl-amino)-ethyl]-1H-quinolin-2-one, 5-[2-(5,6-dimethoxy-indan-2-ylamino)-1-hydroxy-ethyl]-8-hydroxy-1H-quinolin-2-one, 5-[2-(5,6-diethyl-indan-2-ylamino)-1-hydroxy-ethyl]-8-hydroxy-3-methyl-1H-quinolin-2-one, 5-[2-(5,6-diethyl-indan-2-ylamino)-1-hydroxy-ethyl]-8-methoxy-methoxy-6-methyl-1H-quinolin-2-one, 5-[2-(5,6-diethyl-indan-2-ylamino)-1-hydroxy-ethyl]-8-hydroxy-6-methyl-1H-quinolin-2-one, 8-hydroxy-5-[2-(5,6-diethyl-indan-2-ylamino)-1-hydroxy-ethyl]-3,4-dihydro-1H-quinolin-2-one, 5-[(R)-2-(5,6-diethyl-2-methyl-indan-2-yl-amino)-1-hydroxy-ethyl]-8-hydroxy-1H-quinolin-2-one, (S)-5-[2-(4,7-diethyl-indan-2-ylamino)-1-hydroxy-ethyl]-8-hydroxy-1H-quinolin-2-one hydrochloride, 5-[(R)-1-hydroxy-2-(6,7,8,9-tetrahydro-5H-benzocyclohepten-7-ylamino)-ethyl]-8-hydroxy-1H-quinolin-2-one hydrochloride, (R)-5-[2-(5,6-diethyl-indan-2-ylamino)-1-hydroxy-ethyl]-8-hydroxy- 1H-quinolin-2-one maleate, (R)-5-[2-(5,6-diethyl-indan-2-ylamino)-1-hydroxy-ethyl]-8-hydroxy-1H-quinolin-2-one hydrochloride, (R)-8-hydroxy-5-[(S)-1-hydroxy-2-(4,5,6,7-tetramethyl-indan-2-ylamino)-ethyl]-1H-quinolin-2-one, 8-hydroxy-5-[(R)-1-hydroxy-2-(2-methyl-indan-2-ylamino)-ethyl]-1H-quinolin-2-one, 5-[2-(5,6-diethyl-indan-2-ylamino)-ethyl]-8-hydroxy-1H-quinolin-2-one, 8-hydroxy-5-[(R)-1-hydroxy-2-(2-methyl-2,3,5,6,7,8-hexahydro-1H-cyclo-penta[b]naphthalen-2-ylamino)-ethyl]-1H-quinolin-2-one, and 5-[(S)-2-(2,3,5,6,7,8-hexahydro-1H-cyclopenta[b]naph-thalen-2-ylamino)-1-hydroxy-ethyl]-8-hydroxy-1H-quinolin-2-one.

5. A medicament according to claim 4, in which (A) is (R)-5-[2-(5,6-diethyl-indan-2-ylamino)-1-hydroxyethyl]-8-hydroxy-1H-quinolin-2-one maleate.

6. A medicament according to claim 1 wherein the glycopyrronium salt is a racemate or a mixture of diastereomers.

7. A medicament according to 1 wherein the glycopyrronium salt is a single enantiomer.

8. A medicament according to claim 1 wherein the glycopyrronium salt is glycopyrronium bromide.

9. A medicament according claim 6 wherein the glycopyrronium salt is glycopyrronium bromide.

10. A medicament according claim 7 wherein the glycopyrronium salt is glycopyrronium bromide.

11. A medicament according to claim 10 wherein the glycopyrronium salt is (3S,2′R )-3-[(cyclopentyl-hydroxyphenylacetyl)oxy]-1,1-dimethylpyrrolidinium bromide or (3R,2′R)-3-[(cyclopentyl-hydroxyphenylacetyl)oxy]-1,1-dimethylpyrrolidinium bromide.

12. A medicament according to claim 9 wherein the glycopyrronium salt is (3S,2′R/3R,2′S)-3-[(cyclopentyl-hydroxyphenylacetyl)oxy]-1,1-dimethylpyrrolidinium bromide.

13. A medicament according to compound according to claim 1, in which (C) is a compound of formula II where T is a heterocyclic aromatic group having a 5-membered heterocyclic ring with one, two or three ring hetero atoms selected from nitrogen, oxygen and sulfur, the heterocyclic ring being unsubstituted or substituted by one or two substituents selected from halogen, C₁-C₄-alkyl, halo-C₁-C₄-alkyl, C₁-C₄-alkoxy, C₁-C₄-alkylthio, cyano or hydroxy-C₁-C₄-alkyl, and the heterocyclic ring being optionally fused to a benzene ring.

14. A medicament according to compound according to claim 1 in which (C) is a compound of formula II where T is a heterocyclic aromatic group having a 6-membered heterocyclic ring with one or two ring nitrogen atoms, the heterocyclic ring being unsubstituted or substituted by one or two substituents selected from halogen, cyano, hydroxyl, C₁-C₄-acyloxy, amino, C₁-C₄ alkylamino, di-(C₁-C₄-alkyl)amino, C₁-C₄-alkyl, hydroxy-C₁-C₄-alkyl, halo-C₁-C₄-alkyl C₁-C₄-alkoxy, or C₁-C₄-alkylthio and the heterocyclic ring being optionally fused to a benzene ring.

15. A medicament according to compound according to claim 1 in which (C) is a compound of formula II where T is 5-methyl-2-thienyl, N-methyl-2-pyrrolyl, cyclopropyl, 2-furyl, 3-methyl-2-furyl, 3-methyl-2-thienyl, 5-methyl-3-isoxazolyl, 3,5-dimethyl-2-thienyl, 2,5-dimethyl-3-furyl, 4-methyl-2-furyl, 4-(dimethylamino)phenyl, 4-methylphenyl, 4-ethyl-phenyl, 2-pyridyl, 4-pyrimidyl or 5-methyl-2-pyrazinyl or the indicated 16-methyl group has the beta conformation and R is cyclopropyl.

16. A medicament according to compound according to claim 13, in which (B) is 3-methyl-thiophene-2-carboxylic acid (6S,9R, 10S, 11S, 13S, 16R, 17R)-9-chloro-6-fluoro-11-hydroxy-17-methoxycarbonyl-10,13,16-trimethyl-3-oxo-6,7,8,9,10,11,12,13,14,15,16,17-dodecahydro-3H-cyclopenta-[a]phenanthren-17-yl ester.

17. A medicament according to claim 1 further comprising another drug substance selected from an anti-inflammatory, a bronchodilator, an antihistamine, a decongestant or an anti-tussive drug substance.

18. A medicament according to claim 1, which is in inhalable form and is
(i) an aerosol comprising a mixture of (A), (B) and (C) in solution or dispersion in a propellant;
(ii) a combination of an aerosol containing (A) in solution or dispersion in a propellant, with an aerosol containing (B) in solution or dispersion in a propellant and an aerosol containing (C) in solution or dispersion in a propellant;
(iii) a nebulizable composition comprising a dispersion of (A), (B) and (C) in an aqueous, organic or aqueous/organic medium; or
(iv) a combination of a dispersion of (A) in an aqueous, organic or aqueous/organic medium with a dispersion of (B) in an aqueous, organic or aqueous/organic medium and a dispersion of (C) in an aqueous, organic or aqueous/organic medium.

19. A medicament according to claim 1 in which (A), (B) and (C) are present in inhalable form as a dry powder comprising finely divided (A), (B) and (C) optionally together with at least one particulate pharmaceutically acceptable carrier.

20. A medicament according to claim 1, in which (A), (B) and (C) have an average particle diameter up to 10 μm.

21. A medicament according to claim 18, in which (A), (B) and (C) have an average particle diameter up to 10 μm.

22. A medicament according to claim 19, in which (A), (B) and (C) have an average particle diameter up to 10 μm.

23. A medicament according to claim 1, which is
a dry powder in a capsule, the capsule containing a unit dose of (A), a unit dose of (B), a unit dose of (C) and a pharmaceutically acceptable carrier in an amount to bring the total weight of dry powder per capsule to between 5 mg and 50 mg; or
an aerosol comprising (A), (B) and (C) in a propellant, optionally together with a surfactant and/or a bulking agent and/or a co-solvent suitable for administration from a metered dose inhaler adapted to deliver an amount of aerosol containing a unit dose of (A), a unit dose of (B) and a unit dose of (C), or a known fraction of a unit dose of (A), a known fraction of a unit dose of (B) and a known fraction of a unit dose of (C), per actuation.

24. A method of treating inflammatory or obstructive airways disease in a subject in need of such treatment, which method comprises administering to said subject a medicament comprising, separately or together (A), (B) and (C) as defined in claim 1 for simultaneous, sequential or separate administration.

25. The method of claim 24, wherein the disease is asthma or chronic obstructive pulmonary disease.

26. A pharmaceutical kit comprising (A), (B) and (C) as defined in claim 1 in separate unit dosage forms, said forms being suitable for administration of (A), (B) and (C) in effective amounts, together with one or more inhalation devices for administration of (A), (B) and (C).
